(19) United States
(12) Patent Application Publication
(10) Pub. No.: US 2025/0331713 A1
(43) Pub. Date: Oct. 30, 2025

(54) SINGLE DEVICE REMOTE VISUAL ACUITY TESTING SYSTEMS AND METHODS

(71) Applicant: 6 OVER 6 VISION LTD., Tel Aviv (IL)

(72) Inventor: Ofer Limon, Tel Aviv (IL)

(21) Appl. No.: 18/864,642

(22) PCT Filed: Jun. 20, 2023
(86) PCT No.: PCT/IB2023/000374

§ 371 Date: Nov. 11, 2024

Related U.S. Application Data
(60) Provisional application No. 63/353,485, filed on Jun. 17, 2022.

(51) Int. Cl. A61B 3/032; A61B 3/00; A61B 3/14

(52) U.S. Cl. CPC A61B 3/032; A61B 3/0025; A61B 3/0033; A61B 3/005; A61B 3/0058; A61B 3/14

(57) ABSTRACT

Systems and methods for administering a visual acuity test include using a computing device including an image capturing device and a display screen. One or more images of a test subject are captured and used to determine a separation distance between the computing device and the test subject. Identifying data provided by the computing device can be used to determine device characteristics of the image capturing device and display screen so that the separation distance can be estimated and so that graphics (e.g., optotypes) can be produced by the display screen at a size related to the separation distance and to the display screen's characteristics. Parts of a visual acuity exam can be administered as a test subject vocally reads the graphics, their vocal input is recorded, analyzed, and scored. Systems may use object detection algorithms to track whether the test subject has covered an eye with an appendage.

### CROSS REFERENCE TO RELATED APPLICATIONS

This application claims priority to U.S. Provisional Patent Application No. 63/353,485, filed on 17 Jun. 2022 and entitled, “SINGLE DEVICE REMOTE VISUAL ACUITY TESTING SYSTEMS AND METHODS,” the entire disclosure of which is incorporated herein by this reference.

### TECHNICAL FIELD

The present disclosure generally relates to systems and methods for providing self-administered vision tests. More particularly, the present disclosure relates to systems and methods for providing self-administered visual acuity exams using a single device.

### BACKGROUND

A vision test or eye exam is commonly given by an eye doctor to determine whether the patient needs (or needs changes to) prescription lenses such as contact lenses or eyeglasses. The doctor often presents a series of optotypes (which are usually specially-designed letters or numbers) to a test subject who attempts to correctly read each letter or number in the series, and that information is used to determine characteristics of the test subject's vision, often resulting in a prescription or change in prescription for the test subject. This presentation of letters or numbers is commonly known in the art as a visual acuity test or a “refraction” of their eyes. In some cases, specialized equipment may be needed to determine a patient prescription.

Traditionally, visits to the eye doctor and the doctor's examination office have been required prior to obtaining corrective vision lenses or contacts or to receive changes to, or confirmation of, a prescription. A prescription, issued by an eye doctor, has governed the ability to receive corrective lenses and often has been required to be performed at the office of the doctor. These eye examination visits can be costly and time consuming, requiring an individual to take time off work or other obligations to travel to the doctor's examination office for an eye examination.

For this and other reasons, there is a need for improvements in the field of refraction exams that can be more efficient and cost effective for the patient.

### SUMMARY

One aspect of the disclosure relates to a method of testing visual acuity of a test subject using a computing device, the method including: obtaining an image of a test subject via an image capturing device of a computing device, the image including a first physical feature of the test subject and a second physical feature of the test subject; calculating an estimated first size dimension of the first physical feature based on a property of the image capturing device and a first expected size dimension of the first physical feature; calculating an estimated second size dimension of the second physical feature based on the property of the image capturing device and a second expected size dimension of the second physical feature; and determining a separation distance between the test subject and the image capturing device based on the estimated first size dimension and the estimated second size dimension.

In some embodiments, the method further includes displaying a graphic to the test subject using a display screen of the computing device.

In some embodiments, the graphic includes a set of optotypes.

In some embodiments, a dimension of the graphic on the display screen is based on the separation distance.

In some embodiments, the dimension of the graphic is static while displayed on the display screen.

In some embodiments, the dimension of the graphic is dynamically adjusted while displayed on the display screen based on a second determination of the separation distance between the test subject and the image capturing device.

In some embodiments, the first physical feature includes a portion of an eye of the test subject.

In some embodiments, the portion of the eye includes an iris.

In some embodiments, the first expected size dimension is based on a mean size dimension of the first physical feature among test subjects having a characteristic in common with the test subject, or a median size dimension of the first physical feature among test subjects having a characteristic in common with the test subject.

In some embodiments, the characteristic includes at least one of an age, a gender, a sex, a height, a weight, or an ethnicity of the test subject.

In some embodiments, the property of the image capturing device includes a field of view.

In some embodiments, the method further includes detecting the property of the image capturing device by receiving device reference information from the computing device.

In some embodiments, the device reference information includes a field of view and a pixels per inch of an image sensor of the image capturing device.

Another aspect of the disclosure relates to a method of testing visual acuity of a test subject using a computing device, the method including determining a separation distance between a test subject and an image capturing device of a computing device; determining at least one property of a display screen of the computing device; and displaying a graphic to the test subject via the display screen, the graphic having at least one size dimension based on the separation distance and the at least one property of the display screen.

In some embodiments, the graphic includes at least one optotype image.

In some embodiments, the at least one property of the display screen includes a pixels per inch measurement.

In some embodiments, the at least one property of the display screen includes an outer dimension or diagonal dimension of a viewable area of the display screen.

In some embodiments, determining the separation distance includes obtaining an image of a test subject via the image capturing device of the computing device, the image including a physical feature of the test subject; calculating an estimated size dimension of the physical feature based on a property of the image capturing device and an expected size dimension of the physical feature; and determining the separation distance based on the estimated size dimension.

In some embodiments, determining the at least one property of the display screen of the computing device includes receiving the at least one property via a signal transmitted from the computing device.

In some embodiments, the at least one size dimension of the graphic is inversely related to the separation distance.

In some embodiments, the at least one size dimension of the graphic is directly related to the at least one property of the display screen.

In some embodiments, the at least one size dimension of the graphic is inversely related to the at least one property of the display screen.

In some embodiments, the image capturing device and the display screen are positioned in a single housing of the computing device.

In some embodiments, the computing device includes a smartphone, tablet computer, or desktop computer.

Yet another aspect of the disclosure relates to a method of testing visual acuity of a test subject using a computing device, the method including: displaying, via a display screen of a computing device, a first graphical image for a test subject, the first graphical image having a first size dimension; recording, via an input device of the computing device, a response input from the test subject; calculating a test score based on the response input; and displaying, via the display screen, a second graphical image for the test subject, the second graphical image having a second size dimension, the second size dimension being based on the test score.

In some embodiments, the input device includes an audio capturing device.

In some embodiments, the input device includes an image capturing device.

In some embodiments, calculating the test score includes detecting a set of test responses within the response input from the test subject; comparing each test response of the set of test responses to a set of expected test responses to obtain a set of correct test responses; and comparing a number of correct test responses in the set of correct test responses to a total number of test responses.

In some embodiments, the second size dimension is decreased relative to the first size dimension based on the test score exceeding a threshold score value.

In some embodiments, the method further includes recording, via the input device, a second response input from the test subject; calculating a second test score based on the second response input; and calculating a visual acuity measurement of the test subject based on the second test score.

Yet another aspect of the disclosure relates to a method of testing visual acuity of a test subject using a computing device, the method including: obtaining an image of a test subject via an image capturing device of a computing device, the image including a face of the test subject; instructing the test subject, by the computing device, to position an appendage of the test subject relative to the face; obtaining a second image of the test subject via the image capturing device; detecting the appendage of the test subject relative to the face; and displaying to the test subject, by a display screen of the computing device, a graphical image in response to detecting the appendage of the test subject relative to the face. In some embodiments, the appendage is part of an arm of the test subject.

### BRIEF DESCRIPTION OF THE DRAWINGS

The accompanying drawings and figures illustrate a number of exemplary embodiments and are part of the specification. Together with the present description, these drawings demonstrate and explain various principles of this disclosure. A further understanding of the nature and advantages of the present invention may be realized by reference to the following drawings. In the appended figures, similar components or features may have the same reference label.

FIG. 1 illustrates a framework for conducting a visual acuity test;

FIG. 2 illustrates a computing device;

FIG. 3 illustrates a process flow for a visual acuity test;

FIGS. 4-7 illustrate user interfaces for a computing device conducting a visual acuity test;

FIG. 8A illustrates a test subject and measurements thereof;

FIG. 8B illustrates captured images of a physical feature of the test subject of

FIG. 8A.

FIG. 9 illustrates a process flow for a visual acuity test;

FIG. 10 illustrates a process flow for a visual acuity test;

FIG. 10A illustrates a device as it would appear at various separation distances from a test subject;

FIG. 10B illustrates devices having different display screen properties displaying a graphic;

FIG. 11 illustrates a process flow for a visual acuity test;

FIGS. 12-13 illustrate user interfaces for a computing device conducting a visual acuity test;

FIG. 14 illustrates a process flow for a visual acuity test;

FIG. 15A-15B illustrates an image of a test subject with and without an appendage covering the test subject's face;

FIG. 16 illustrates a user interface for a computing device conducting a visual acuity test;

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

Embodiments of the present disclosure relate to a visual acuity test including several stages of a test process. Each stage can include at least one specifically engineered graphical image, which can be a series of letters or numbers, which can be referred to as optotypes. The optotypes shown on the computing device display screen can be presented to a user (i.e., a test subject, patient, or similar individual being tested). The optotypes can appear different to the subject based on the physical characteristics of their eyes, such as visual acuity, their distance from the display screen presenting the optotypes, or similar factors. In each stage, the test process can be used to determine aspects of the visual acuity of the subject based on measurements of and/or calculations of a distance along an optical path from the target to the test subject, based on changing the graphical image or optotype dimensions or symbols, and/or based on what the test subject is actually seeing based on their input (e.g., verbal responses or other user input/data received by the computing device). Taken together, these parameters can be used to determine details of the visual acuity of the subject. In some embodiments, the test process can include instructing the subject via visual and audio cues to guide the user through the exam. In some configurations, the exam is performed for each eye separately, while covering the opposite eye, so the test subject performs the test twice. In some embodiments, the test is administered binocularly and monocularly, with separate monocular tests for each eye.

The test process can be self-administered and performed using a single device, meaning the test subject can be the user operating the computing device and implementing the test process without needing a separate external display, a non-user calibration object, a separate measurement device, or similar objects. In some embodiments, the test subject can therefore be alone or receive no assistance from other nearby people while completing the test process. The results of the test process can, in some cases, be provided to a third party (e.g., an eyecare professional) to interpret the results and take appropriate action. Information gathered using the test process (e.g., the subject's visual acuity score) can be used to provide the subject with information about their eyesight. For example, the visual acuity score measurements of the test subject can be used to formulate a new or updated prescription for corrective lenses (glasses and/or contact lenses) for the test subject.

In at least one embodiment, a method and system testing visual acuity of a test subject using a computing device is disclosed. The method can include determining a distance of a test subject from the computing device by obtaining images of a first physical feature and a second physical feature of the test subject. The dimensions of the physical features can be calculated based on a first and second expected size of the physical feature. A separation distance between the first and second dimensions can then be calculated.

In another embodiment, a method of testing visual acuity of a test subject using a computing device includes determining a separation distance between a test subject and an image capturing device, determining at least one property of a display screen of the of the computing device, and displaying a graphic to the test subject based on the separation distance and the at least one property of the display screen.

In at least one embodiment, a method of testing visual acuity of a test subject using a computing device is disclosed, wherein a display screen, which can be disposed on an electronic device, can display a first graphical image for a test subject having a first size dimension. The test subject can provide a response input to the first graphical image that can be recorded by an input device of the computing device, and the response input can be analyzed or calculated to determine a test score based on the test subject's response input. The display screen can display a second graphical image for a test subject having a second size dimension responsive to the test subject's response input or test score. The test subject can provide a second response input to the second graphical image that can be recorded by an input device of the computing device, and the response input can be analyzed or calculated to determine a second test score based on the test subject's second response input. In another embodiment, a method is demonstrated related to determining the size and scale of the test images or optotypes based on referencing device identifying information and the between the test subject and device.

In another embodiment, a method of testing visual acuity of a test subject using a computing device is disclosed which can include capturing an image using the computing device which can include a face of the test subject and instructing the test subject to position a body part or appendage of the test subject, such as their hand or forearm, over the test subject's face. A second image can be obtained to via the image capturing device, which can be disposed on the computing device, to detect the appendage of the test subject relative to the face. Based on the two images, a display screen disposed on the electronic computing device can provide a graphical image in response to detecting the appendage of the test subject relative to the subject's face.

The present description provides examples, and is not limiting of the scope, applicability, or configuration set forth in the claims. Thus, it will be understood that changes may be made in the function and arrangement of elements discussed without departing from the spirit and scope of the disclosure, and various embodiments may omit, substitute, or add other procedures or components as appropriate. For instance, the methods described may be performed in an order different from that described, and various steps may be added, omitted, or combined. Also, features described with respect to certain embodiments may be combined in other embodiments.

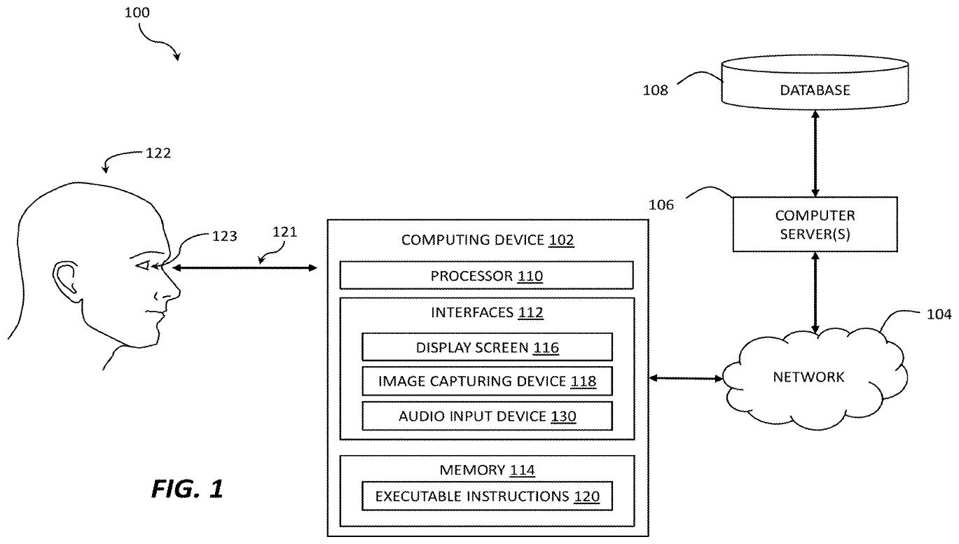

FIG. 1 illustrates an exemplary system or framework 100 for conducting a visual acuity eye examination with a single device (e.g., a single computing device 102 that performs all test functions and calculations or a single testing device at the user's location that only communicates with other remote/networked non-user-controlled computing devices). This and other arrangements and elements (e.g., machines, interfaces, function, orders, and groupings of functions, etc.) can be used in addition to or instead of those shown, and some elements may be omitted altogether. Further, many of the elements described may be implemented as discrete or distributed components or in conjunction with other components, and in any suitable combination and location. Various functions described herein as being performed by one or more components may be carried out by firmware, hardware, and/or software. For instance, and as described herein, various functions may be carried out by a processor executing instructions stored in memory.

Among other components not shown, framework 100 of FIG. 1 includes at least one network 104, at least one computer server 106, at least one database 108, and at least one computing device 102. Computing device 102 can include processor 110, interfaces 112, and memory 114. Memory 114 includes (e.g., may be encoded with) executable instructions 120 for performing a visual acuity test. The memory 114 can include a non-transitory computer-readable medium having executable instructions 120 stored therein or encoded thereon. The interfaces 112 can include at least one visual display screen 116 (among other potential output devices such as an audio output device/speaker) and at least one image capturing device 118 (among other potential input devices such as an audio input device 130/microphone or capacitive touch sensor for the display screen 116). The image capturing device 118 can capture an image (e.g., photograph) or series of images (e.g., photographs or videos). It should be understood that framework 100 shown in FIG. 1 is an example of one suitable framework for implementing certain aspects of this disclosure. Additional, fewer, and/or different components may be used in other embodiments. It should be noted that implementations of the present disclosure are equally applicable to other types of devices such as mobile computing devices and devices accepting gesture, touch, and/or voice input (e.g., via audio input device 130). Any and all such variations, and combinations thereof, are contemplated to be within the scope of implementations of the present disclosure. Further, although illustrated as a computing device 102, and a number of components can be used to perform the functionality described herein.

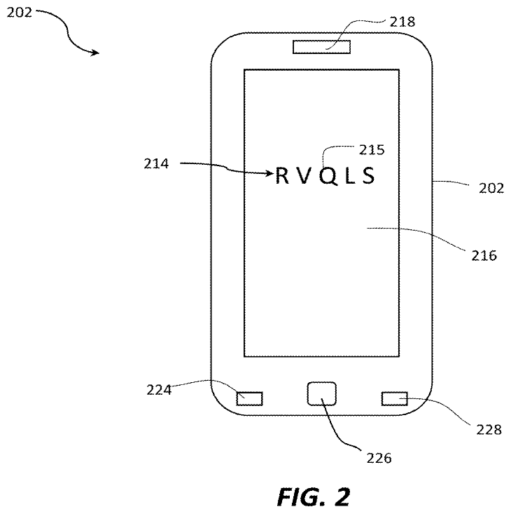

As shown in FIG. 1, computing device 102, display screen 116, and image capturing device 118 may electronically communicate directly with each other via an electronic bus (or related interfaces known in the art) to a processor 110 which can be prompted to perform actions by the executable instructions 120 stored or encoded therein on memory 114. The computing device 102 and the interfaces 112 may have access (e.g., via network 104) to the at least one computer server 106 and the database 108, which may include any data related to prescription data, refraction data, visual acuity measurements, user data, size data, historical data, comparative data, as well as any associated metadata therewith. Computer servers 106 and database 108 may further include any data or related techniques or executable instructions for performing a visual acuity test process using a graphical image 214, as shown in FIG. 2, such as a series of letters or numbers such as optotypes, to present to a test subject 122, instructions for the test subject 122, product properties, control signals, and indicator signals. In implementations of the present disclosure database 108 may be searchable for its data and techniques or executable instructions described herein. Additionally, database 108 may include a plurality of unrelated data repositories or sources within the scope of embodiments of the present technology. Database may be local to the computing device 102. Database 108 may be updated at any time.

The display screen 116 can interface with the computing device 102. The display screen 116 can be used to display images to the test subject or other user of the framework 100. In some embodiments, the display screen 116 can include an electronic display (e.g., a liquid crystal display (LCD), e-ink display, image projector, or similar device). The display screen can be used to present a plurality of letters and/or numbers to a test subject 122, such as optotypes to evaluate the subject's refraction or visual acuity, instructions on how to conduct the test, or information such as test results. The test subject can view images on the display screen 116 and provide input to the computing device 102 concerning their perception (e.g., letter or number) of the optotypes. Based on the feedback from the test subject, the display screen 116 can be controlled to present different graphical images (e.g., 214), which can be a series of optotypes, to the test subject to evaluate eyesight and to assist in determining their level of visual acuity.

Examples of the image capturing device 118 may include sensors configured to collect image information. In some embodiments, the image capturing device 118 may be part of the computing device 102, such as being located within a housing of the computing device that also contains the display screen 116. In some embodiments, the computing device 102 is a mobile computing device, such as a smart phone device or tablet computer configured with a camera as the image capturing device 118. In some embodiments, the image capturing device 118 includes a plurality of image capturing devices capable of collecting image data. In some embodiments, the image capturing device 118 can be used to obtain an image of the user, the user's eyes, or other objects, or multiple image capturing devices can be used to obtain different images. The image capturing device 118 can be configured to capture an image of the test subject while the test subject faces the display screen 116, such as by being a front-facing camera. In some embodiments, the image capturing device 118 can receive input from the test subject 122 (e.g. by the test subject looking in a certain direction, performing a gesture, focusing on a series of optotypes or a particular optotype, etc.), which may be at a separation distance 121 from the computing device 102. Examples herein may include computing devices, such as computing devices 102 of FIG. 1. Computing device 102 can include additional interfaces 112 such as sensors (e.g., a display screen 116, an image capturing device 118, microphones, keyboards, speakers, and other input devices) described herein.

Computing devices, such as computing device 102 described herein may include one or more processors, such as processor 110. Any kind and/or number of processor may be present, including one or more central processing units(s) (CPUs), graphics processing units (GPUs), other computer processors, mobile processors, digital signal processors (DSPs), microprocessors, computer chips, and/or processing units configured to execute machine-language instructions and process data, such as executable instructions 120. A computing device 102 can also include other computer components (not shown) to operate and interconnect the computing device 102, such as, for example, an input/output controller (I/O), a display or other output device, input device, network interfaces, etc.

Computing devices, such as computing device 102, described herein may further include memory 114. Any type of memory may be present (e.g., read only memory (ROM), random access memory (RAM), solid state drive (SSD), secure digital card (SD card, similar devices, and combinations thereof). While a single memory 114 is shown in FIG. 1, any number of memory devices may be present. The memory 114 may be in electrical communication (e.g., electrically connected) with the processor 110.

Memory 114 may store executable instructions for execution by the processor 110, such as executable instructions 120 for determining visual acuity of the test subject's eye 123. Processor 110, being communicatively coupled to image capturing device 118 and display screen 116, and via the execution of executable instructions 120 for determining visual acuity, may track test subject information and changes based on data collected form the image capturing device 118, among other input devices or interfaces 112.

In some embodiments, a self-administered visual acuity test is used to determine the visual acuity of each or both of the test subject's eyes. The exam may include a combination of several sequential or parallel steps, some of which may include presenting a graphic 214 via a display screen 216, as illustrated in FIG. 2. The graphic 214 can include letters, numbers, symbols, glyphs, patterns, shapes, similar images, or combinations thereof. Specific optotypes 215 that are part of the graphic 214 may have a visually different appearance relative to other optotypes in the graphic 214 based on the test subject's visual acuity, their distance from the optotypes, and display contrast, colors, resolution, and size. In various stages of a test, a series of optotypes 215, as illustrated in FIG. 2, is presented with the optotype size differing based on the specific test stage, the distance 121 between the image capturing device/display screen and the test subject 122, or the number of optotypes correctly identified by the test subject in audible responses to the series of optotypes presented. A test score, which can include a visual acuity score, can then be calculated based on the subject's verbal responses compared to the optotypes presented on the display screen 116 of computing device 102. The exam can be performed for each eye separately (e.g., while covering one eye at a time) so the subject can perform the same or similar portions of the test at least twice. The exam can also be performed for both eyes at the same time, with neither of the subject's eyes being covered during that portion of the test. The exam can be performed with a combination of the subject's eyes being covered or uncovered, so the subject can perform the same or similar portions of the test at least twice.

FIG. 2. illustrates an exemplary computing device 202, such as a mobile computing device (e.g., tablet computer or a smart phone) which may present one or more vision test procedures or portions to a test subject 122 (e.g., a human subject). In some embodiments, the computing device 102 may be, for example, a gaming device, desktop computer, other multimedia device, similar devices, or combinations thereof. The computing device may include a display screen 216 which can be a display screen (e.g., a liquid crystal display (LCD), e-ink display, image projector, or similar device) capable of presenting a graphic 214, which can include one or more optotypes 215, an image capturing device 218, which can be a camera or similar light sensor (including, for example, at least a lens, image sensor, and camera circuitry), an audio capturing device 224 (e.g., microphone) which permits collection of sounds made by the subject 122 (e.g., speech), and a transducer 228 capable of projecting audio, such as a loudspeaker. The computing device can also include a button 226 or other interface device (e.g., a capacitive touch sensor array that is integrated with the display screen 216).

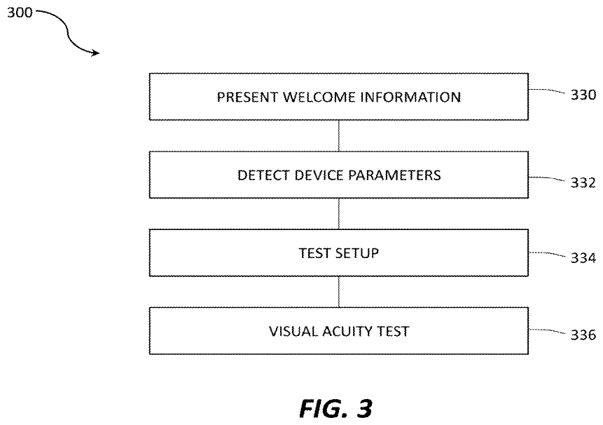

FIG. 3 illustrates an exemplary test flow diagram of a process 300 of an exemplary approach to carry out a visual acuity test with a computing device (e.g., 202). Steps, blocks, actions, and tests described in connection with FIG. 3 can be administered, skipped, or omitted, and it should be understood that sections of the process shown in FIG. 3 are optional.

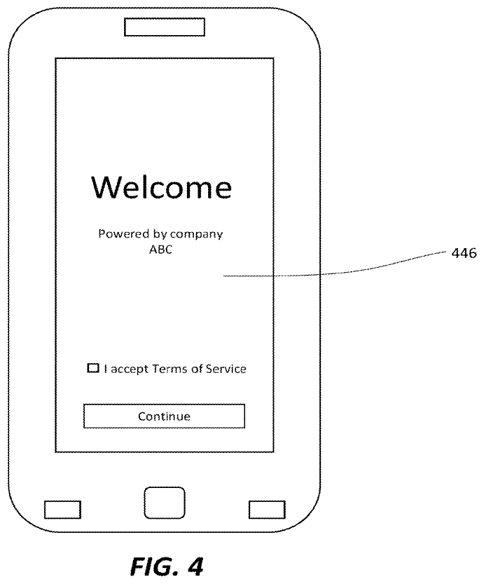

As shown at block 330, the test subject can be presented welcome information, which can include a welcome screen as shown in FIG. 4. The welcome screen shown in exemplary graphical user interface (GUI) 446. The GUI 446 can present company information such as a questionnaire, terms and conditions, a continuation or acceptance panel or other information. The welcome GUI 446 may address various user inclusion/exclusion requirements prior to taking the visual acuity test.

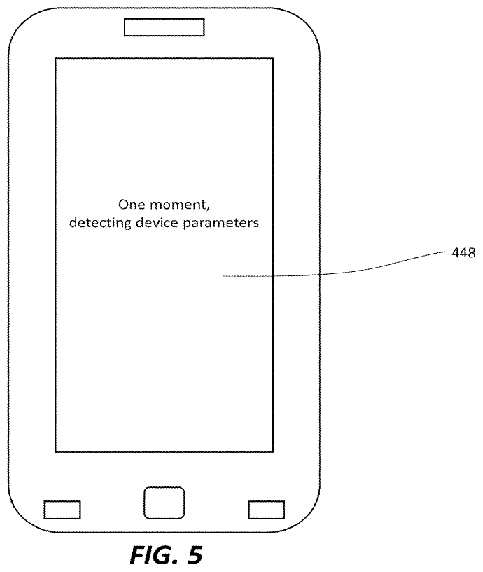

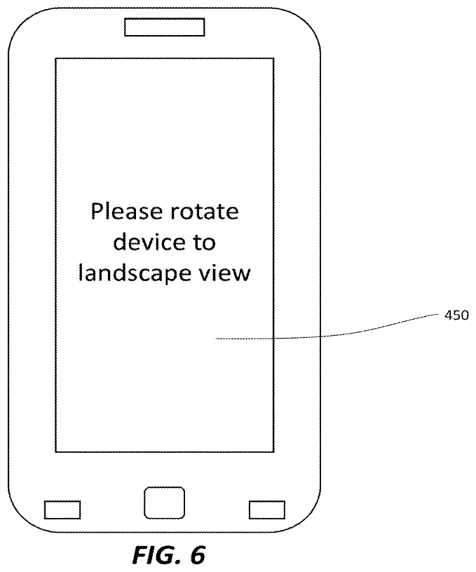

Block 332 can detect the device parameters and properties, as depicted in exemplary GUI 448 of FIG. 5. In block 332, the controller or processor may determine a property of the display screen 216 through the database 108. The property of the display screen 216 can include a pixels per inch measurement, a screen resolution, a screen dimension (e.g., diagonal width), or similar property that affects the size images are displayed to a user via the display screen. This measurement can vary from device to device. A database (e.g., a whitelist of electronic devices) can be stored of devices compatible with the application and their related properties. Other properties related to the computing device, such as properties of the image capturing device 118, can be detected or determined as well, such as by determining a field of view, sensor resolution or sensitivity, or similar properties of the image capturing device. Upon receiving the device display properties and imaging device properties, the controller may adjust settings for the visual acuity test, such as by adapting the size, shape, and type of optotypes presented to a test subject based on the size and resolution of the display screen or by adapting a distance estimating procedure to appropriately estimate a distance between the test subject and the image capturing device based on the field of view and resolution of the image capturing device. Furthermore, if the computing device 202 is not recognized or identified in the database, the user may receive an alert that the computing device 102 is not supported or that certain parameters could not be obtained. In some embodiments, the user can be instructed to input necessary device parameters or to confirm detected or estimated parameters. Upon determining device parameters, instructions (a video) may be shown via the display prompting the user to rotate the device 202 to landscape or horizontal view, as shown in exemplary GUI 450 of FIG. 6. The computing device 202 may incorporate a sensor, such as an accelerometer or other gravity detecting sensor, which can automatically detect the orientation of the computing device 102, to determine whether this instruction is necessary.

Block 334, test setup, can include a video prompting the user to increase the device 102 volume and various directions for the user to follow prior to starting the visual acuity test.

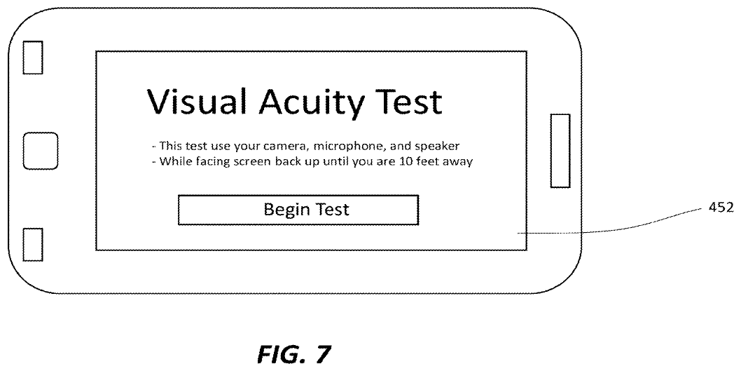

In block 336, the process 300 can include presenting information and directions related to the test parameters and test requirements which are illustrated in the exemplary GUI 452 of FIG. 7. A user may select or follow a prompt to proceed with the visual acuity test. The prompt may be a button, countdown, or other similar test initiation procedure. The test may begin automatically, using the image capturing device 218 to detect the user's location relative to computing device 202.

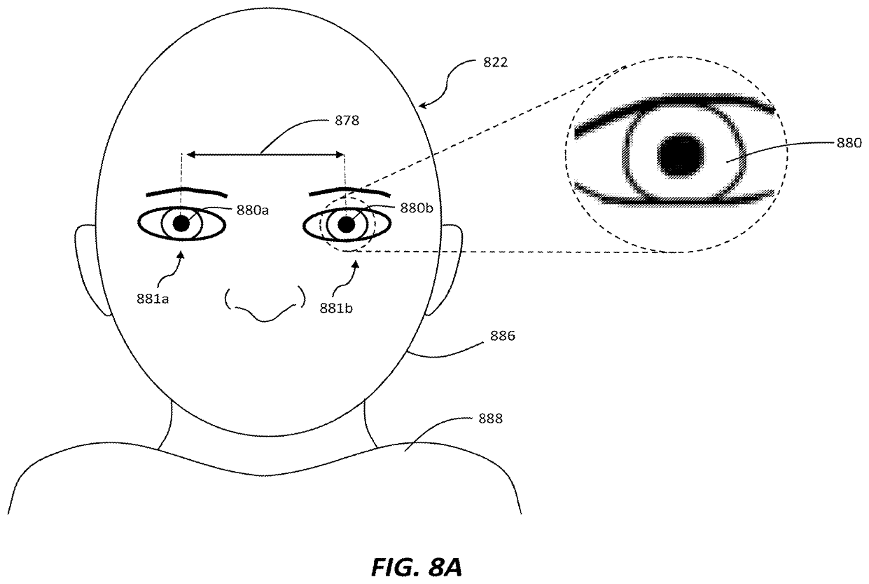

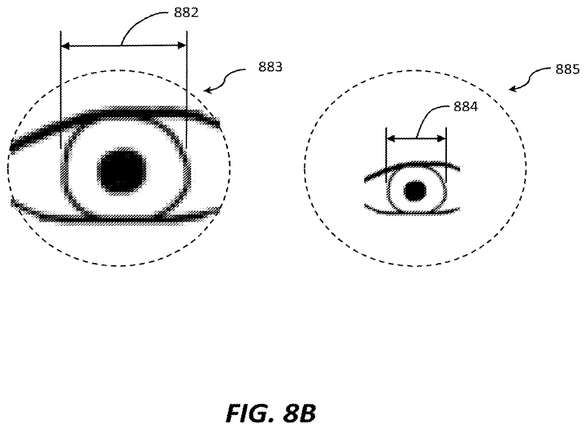

FIG. 8A illustrates a schematic view of an exemplary test subject 822. The image capturing device 218 may capture an image of aspects of the subject's physical features, such as his or her the inter-pupillary distance (IPD) 878, a width of an eye 881a, 881b, a diameter of an iris 880a, 880b of the test subject 822, the location of the subject's head 886 relative to the subject's shoulders 888, or the width of his or her nose, mouth, ears, shoulders, neck, or other user physical features that can be captured by the image capturing device 218. In some embodiments, the image captured can include various physical features of the test subject (i.e., ears, eyes, nose, shoulder, hands, arms, etc.).

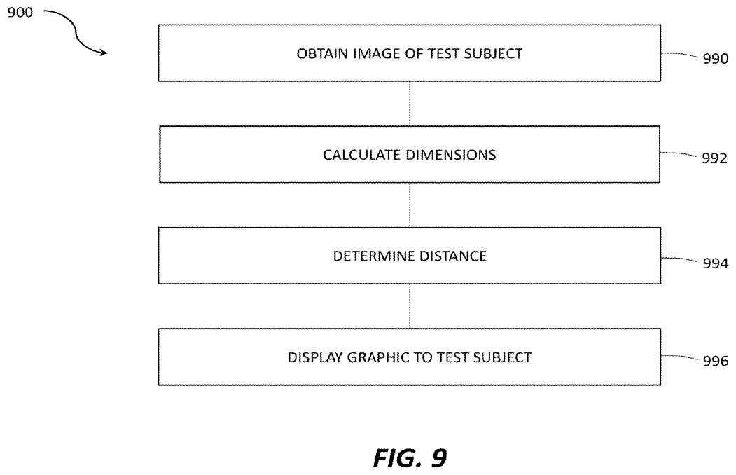

In one exemplary embodiment, a visual acuity test can be implemented using the process 900 illustrated in FIG. 9, which can include block 990, in which the controller can capture an image of a test subject, block 992, in which the controller can calculate dimensions of a feature of the test subject in the captured image, block 994, in which the controller can determine or estimate a separation distance between the image capturing device and the test subject, and block 996, in which the controller can display a graphic to a the test subject (e.g., via a display screen of the computing device).

Block 990 can include obtaining an image of a test subject 822 via the image capturing device 218 of the computing device 202. The image capturing device 218 can have a property such as a field of view. Information regarding the field of view or other property can be received as reference information from the computing device 202. For example, the computing device 202 can have stored identifying information (e.g., in memory 114) that is readable by the processor (e.g., 110). In some embodiments, the identifying information can identify a maker and model of the computing device 202, such as an identification of the manufacturer, the model number, the serial number, the software being used by the computing device (e.g., operating system, application, or software version), or the manufacturer or model of component parts of the computing device 202 (e.g., the maker or model of the display screen 116 or the image capturing device 118), such as in cases where the computing device 202 is an assembly of parts made by multiple manufacturers. By referencing this identifying information, the controller/processor (e.g., of device 102 or server 106) can determine the properties of the image capturing device 218 or display screen 116. For example, a camera's field of view and image sensor resolution can be identified by referencing the camera's make and model and then cross-referencing that information to a database of field of view and sensor resolution data (e.g., in a database 108). Similarly, a display screen's resolution and actual dimensions can be determined by identifying the manufacturer and model of the computing device (e.g., 102) and cross-referencing that information with associated device information (e.g., in a database 108).

The image captured by the image capturing device in connection with block 990 can include a depiction or representation of a first physical feature of the test subject 822 and a second physical feature of the test subject 822 within the same captured image. In some embodiments, the physical features can include the subject's eyes, ears, eyebrows, nostrils, or any other physical feature of the test subject 822 that is visible to the image capturing device 218. In one implementation of the process 900, the physical feature can include a portion of an eye 881 of the test subject, wherein the eye 881 may include an iris 880. In one embodiment, block 990 can include obtaining the property of the image capturing device 218 which can be received as reference information from the computing device by the controller. The device reference information can include the field of view and pixels per inch of an image sensor of the image capturing device 218.

The controller can calculate an estimated first size dimension from of the first physical feature based on a property of the image capturing device 218 and a first expected size dimension of the first physical feature in connection with block 992. An exemplary dimension can be, but is not limited to, interpupillary distance (IPD) 878, a width or height of the eyes 881a, 881b, a width or height (e.g., diameter) of the irises 880, a distance between the eyes 881a, 881b, a width of the nose, etc. The controller can also calculate an estimated second size dimension of the second physical feature based on a property of the image capturing device 218 and second expected size dimension of the second physical feature in block 992. The first physical feature's expected size dimension can be based upon a mean, median, or other expected representative size dimension of the first physical feature among test subjects having a characteristic in common with the test subject.

The common characteristics of the test subjects can include at least one of: an age or age range, a gender, a sex, a height or height range, a weight or weight range, an ethnicity, or other distinguishing, observable, physical characteristic of the test subject. Thus, for example, the controller can estimate the size of the test subject's iris diameter, for each individual eye, based on (1) referencing the test subject's personal information (e.g., age, gender, weight), (2) determining a typical (e.g., mean or median) iris diameter for a typical person having that personal information (e.g., age, gender, weight) using a database of demographic information, medical records, or similar compilations of typical physical feature sizes and positions, and (3) estimating that the test subject has that typical iris diameter or an iris diameter within a small range of variation from that typical diameter. In some embodiments, the physical feature that is targeted and that has its dimensions estimated is a physical feature that has low variation across a large range of the general population that has characteristics in common with the test subject. For example, iris diameter can be beneficially used because that dimension is not affected by a test subject's weight, musculature, makeup, personal grooming, or height as much as some other characteristics (e.g., eyebrow size, ear-to-ear distance, shoulder size, etc.). Iris diameter can also be a useful physical feature since the test subject faces the image capturing device with their eyes open and because iris diameters for a large majority of test subjects will be within a small range of variations (e.g., within about 10 percent of a mean or median value).

In block 994, the controller can determine a separation distance 121 between the test subject 122 and the image capturing device 218 based on the estimated first size dimension and the estimated second size dimension. To do so, the controller can detect the test subject's physical features in the captured image, such as by detecting the left and right iris of the test subject using an object or face detection algorithm or logic. The physical features can have a size in the image (e.g., a width in pixels). The size of the features in the image can be assigned the estimated size dimension determined in connection with block 992. The controller can then also estimate the separation distance 121 by calculating how far away from the image capturing device the test subject would need to be for the physical features to have the pixel sizes appearing in the captured image. This estimated distance calculation can be used as the separation distance 121. This distance calculation can be based on the field of view and sensor resolution of the image capturing device. Cameras having a wider field of view (e.g., that use more “fisheye”-like lenses) can capture images with pixel widths of physical features that are smaller than cameras with a narrower field of view. Similarly, cameras having higher sensor resolution (i.e., higher megapixels of detail captured) can capture images with pixel widths of physical features that are greater than cameras with lower sensor resolution.

In some embodiments, multiple physical features are referenced and have their sizes estimated (e.g., two iris diameters or an iris diameter and an IPD). By using multiple dimensions of reference in a captured image, the controller can have reduced measurement error in the estimates of those dimensions when using the methods described herein.

In block 996, the controller can display a graphic 214 to the test subject 822 using a display screen 216 of the computing device 202. The graphic 214 displayed on the display screen 216 can include a set of optotypes 215, wherein a dimension of the graphic 214 on the display screen 216 is based on the separation distance 121 of the test subject 122 and the computing device 202. For example, the dimension of the graphic can be a width dimension and/or height dimension of the optotypes that is calculated dependent upon the separation distance. A ratio can be applied to the separation distance to establish the dimension of the graphic, wherein the size of the graphic is directly proportional to the separation distance.

In one embodiment, the dimensions of the graphic 214 can be static while displayed on the display screen 216, such that the graphic 214 does not move or change on the display screen 216 in reaction to movement of the test subject 122 changing their original positon or location to a different position or location relative to the computing device 202.

In another embodiment, the dimension of the graphic 214 can be dynamically adjusted while displayed on the display screen 216 based on a second determination of the separation distance between the test subject 122 and the image capturing device 218. The dimension of the graphic can therefore adapt periodically (or in real-time) to the position of the test subject relative to the computing device. This can help increase reliability of the results of the visual acuity test since a test subject is less likely to have their position intentionally or unintentionally drift during the test in such a manner as to make it likely that their ability to read the optotypes is affected (positively or negatively).

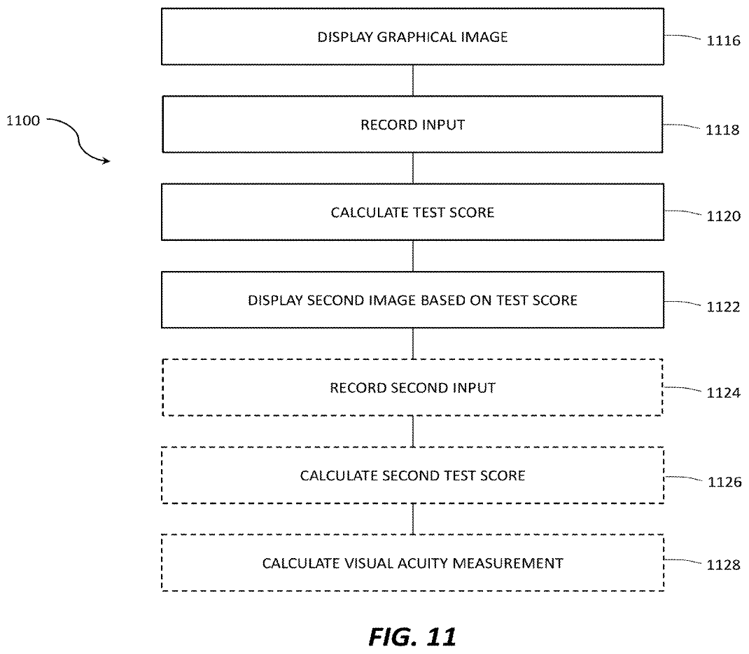

With the graphic being displayed to the test subject in block 996, the test subject can then view the graphic and provide input responses to prompts initiated by the controller, such as vocally reading the optotypes. The subject's responses can be analyzed and can guide the controller to display different graphics over time. In the end, the controller can estimate the test subject's visual acuity based on the accuracy of their input responses, as further explained in connection with FIG. 11 below.

FIG. 8B shows how, in one embodiment, a first image 883 of the test subject that is captured at a first separation distance relative to the computing device 102 can be compared to a second image 885 taken at a second separation distance relative to the computing device 102. Aspects and features of the physical features of test subject can be compared in the first and second images 883, 885. For example, changes in features, ratios, or other aspects of the image 883, 885 size, of the first image 883 relative to the second image 885. The comparison can also include a comparison of changes in features, ratios, or other aspects of the images 883, 885 size, the second image 885 relative to the first image 883.

An image of a physical feature, which can include a first or second physical feature, can be, but is not limited to, a first diameter 882 of the iris 880 of one or both eyes of the test subject 822 and a second diameter 884 of one or both eyes of the test subject 822. The first diameter 882 of the iris 880 of one or both eyes of the test subject can be imaged at a first distance, and the second diameter 884 of one or both eyes of the test subject 822 can be imaged at a second distance. The first image 883 of the first diameter 882 of the iris 880 of one or both eyes can be compared to the second image 885 of the second diameter 884 of the iris 880 of one and both eyes, the first image 883 including an image with the first iris diameter 882 differing the second image 885 of the second iris diameter 884. Thus, the controller can determine that the test subject is at a first separation distance when the diameter 882 is larger (as in image 883) or at a second, greater separation distance when the diameter 884 is smaller (as in image 885).

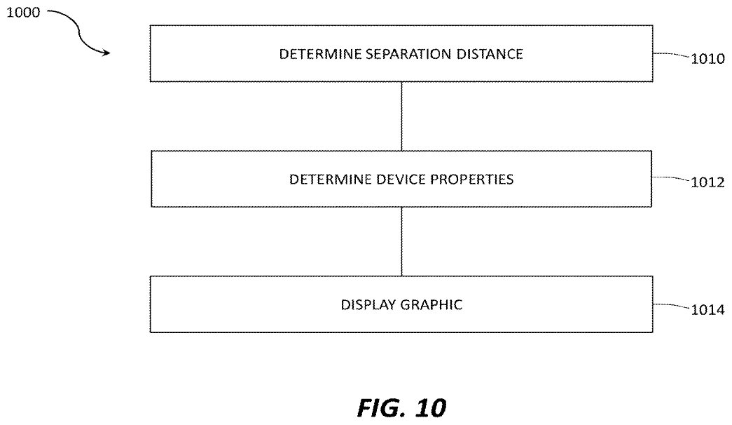

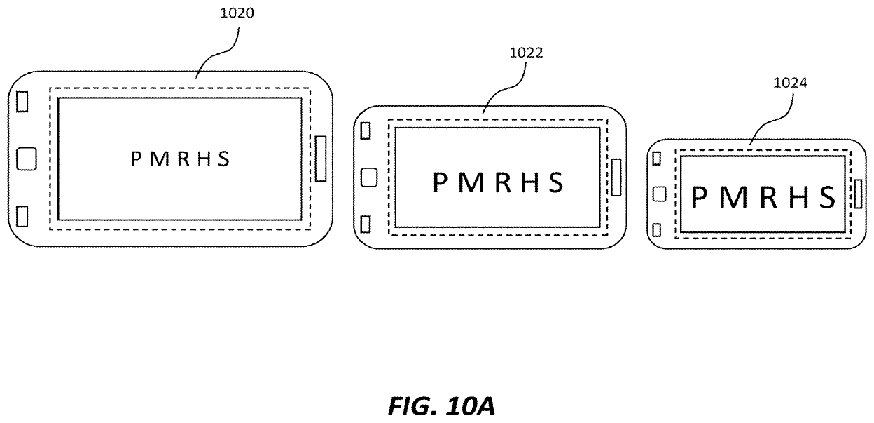

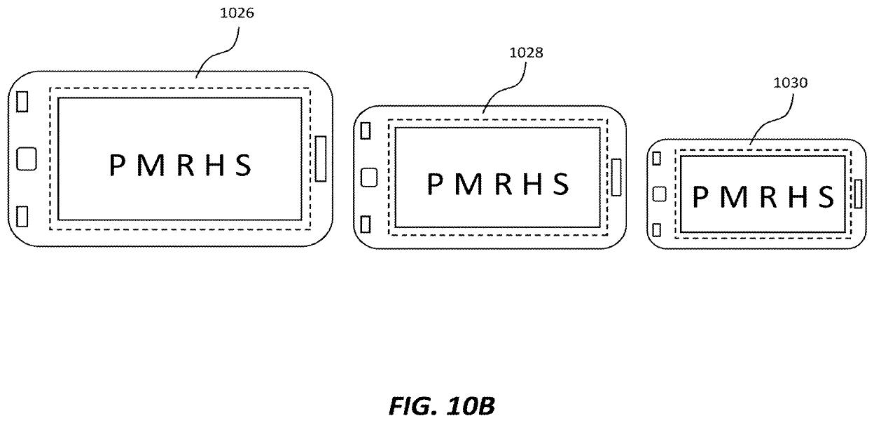

FIG. 10 illustrates a flow diagram of a process 1000 for administering a visual acuity test to a test subject 822 using a computing device 202. The process 1000 can include block 1010, in which a controller can determine a separation distance, block 1012, in which a controller can determine device properties of the computing device, and block 1014, in which the controller can display a graphic to the subject on a display screen of the computing device 202.

In block 1010, the controller can determine a separation distance 121 between a test subject 122 and an image capturing device 118 of a computing device 102. Determining the separation distance can include obtaining an image of the test subject 122 via the image capturing device 118 of the computing device 102, which can include a physical feature, such as the eye 123 of the of the test subject 122, calculating an estimated size dimension of the physical feature based on a property of the image capturing device and an expected size of the physical feature based on a property of the image capturing device and an expected size dimension of the physical feature, and determining the separation distance based on the estimated size dimension. For example, the controller can implement the procedures described in connection with block 994 above.

In block 1012, the controller can determine at least one property of a display screen 216 of the computing device 202. At least one of the properties of the display screen can include a pixels per inch measurement, a screen resolution, a screen dimension, a similar property, or combinations thereof. In some embodiments, an outer dimension or diagonal dimension of a viewable area of the display screen can be determined. The display screen property or properties of the computing device can include receiving the property or properties via a signal transmitted from the computing device. For example, the property or properties can be determined as discussed above in connection with blocks 990 and 992.

In block 1014, the controller can display a graphic 214 to the test subject 122 via the display screen 216, with the graphic having at least one size dimension based on the separation distance 121 and at least one property of the display screen 216. The graphic displayed can include at least one optotype 215 image, which can include a letter or a number. The size dimension of the graphic can be directly related to (e.g., screen size dimension) to the property of the display screen 216 or inversely related (e.g., pixel density/PPI) of the display screen 216. For example, the graphic dimensions can be determined and displayed as discussed above in connection with block 996.

Thus, as shown in FIG. 10A, the graphic can vary between different sizes based on the separation distance and the properties of the computing device display screen. Each device 1020, 1022, 1024 can represent the same device shown at various distances from the test subject. At a first determined separation distance (e.g., 8 feet), the device 1020 can produce relatively small optotypes, at a second separation distance (e.g., 10 feet), the device 1022 can produce relatively larger optotypes, and at a third separation distance (e.g., 12 feet), the device 1024 can produce even larger optotypes, all of which can be sized so as to appear substantially the same size to the test subject at those various separation distances. Furthermore, in some embodiments, as the user moves between different separation distances during the test, the device can adapt the graphic size to ensure consistency of testing conditions.

In another example, as shown in FIG. 10B, three different devices 1026, 1028, 1030 having different display properties (i.e., screen dimensions) are shown displaying the same optotype graphics. The dimensions of the graphics are visually identical on each device 1026, 1028, 1030 even though the display screens have different sizes. Thus, the controller can calculate a proper scale and proportions of the graphic based on the display properties for each device 1026, 1028, 1030 to ensure visual consistency for test subjects, no matter what type of screens their devices have. Additionally, in some embodiments, the devices 1026, 1028, 1030 may have different display screen pixel densities, pixels per inch, resolutions, etc. The controllers of the devices 1026, 1028, 1030 can account for those inconsistencies between devices to accommodate for the variations and scale the graphics as needed to ensure each graphic appears with the desired actual size dimensions as viewed by the user at various distances.

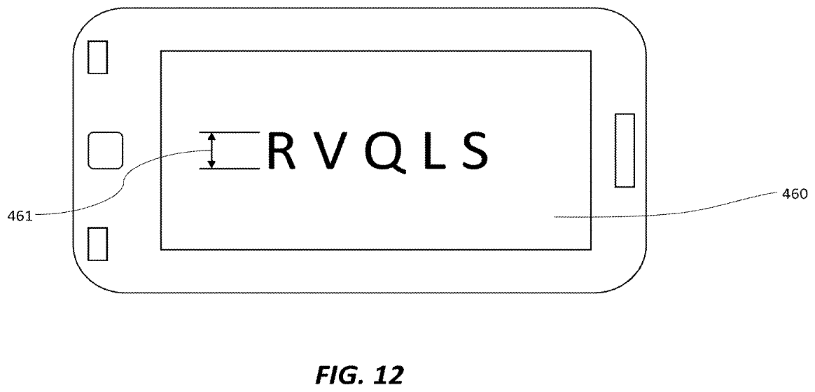

An example process 1100 of administering a visual acuity test is shown in FIG. 11. In block 1116, the controller can display, via a display screen (e.g., 215), a first graphical image, as shown, for example, in GUI 460 of FIG. 12. In block 1118, the controller can record input on the computing device 202 from the test subject. In block 1120, the controller can calculate a test score based on the response input, and in block 1122, the controller can display, via the display screen, a second graphical image, as shown in GUI 462 of FIG. 12.

In block 1116, the controller can display, via a display screen 216 of a computing device 202, a first graphical image 460 to a test subject 122. The first graphical image 460 can have a first size dimension, such as optotype height 461.

In block 1118, the controller can record, via an input device of the computing device, a response input from the test subject. The input device can include an audio capturing device 224, such as a microphone, and/or an imaging capturing device 218, such as a camera. For example, the controller can prompt the test subject to vocally read the graphical image 460 or provide other gestural movements while the computing device records the test subject's vocal or gestural response input.

In block 1120, the controller can calculate a test score based on the response input. The test score calculation can include detecting a set of test responses within the response input recorded from the test subject, comparing each test response of the set of test responses to a set of expected test responses to obtain a set of correct responses, and comparing a number of correct test responses in the set of correct test responses to a total number of test responses. For instance, when five optotypes are displayed on the computing device, the test subject can be recorded reading five letters aloud. A voice/word recognition algorithm can be applied to the recording, as known in the art, and the controller can thereby determine whether the test subject correctly read aloud each letter. Thus, in some embodiments, the test score of block 1120 can include or be based on the number of correctly recorded responses.

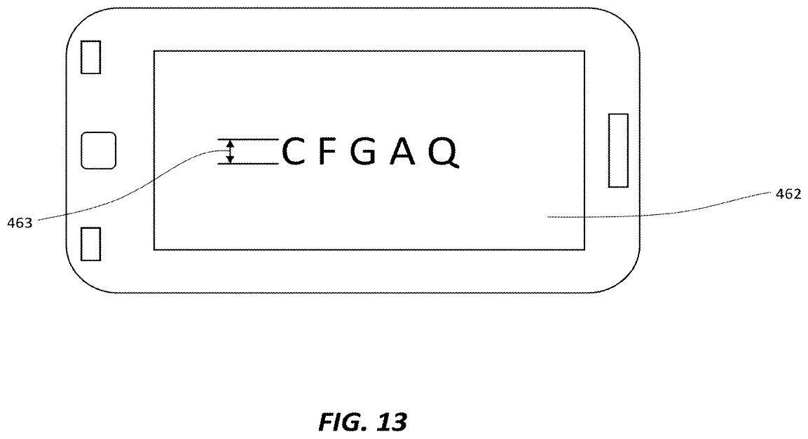

In block 1122, the controller can display, via the display screen 216, a second graphical image 462 for the test subject. The second graphical image 462, shown in FIG. 13, can have a second size dimension 463 which can be based at least partially on the test score. In some instances the second size dimension 463 can be decreased relative to the first dimension based on the test score exceeding a threshold score value. For example, the second size dimension 463 can be decreased if the test subject correctly reads a majority of the symbols displayed on the display screen since the subject's test score indicates that he or she may be capable of reading smaller, more difficult-to-read optotypes at the same testing separation distance from the computing device. The test subject 122 can optionally respond to the second graphical image 462 displayed on the display screen 216 of the electronic device, and the computing device 202 can record, via the input device, a second response input (in a manner similar to block 1118), as indicated in block 1124, and calculate a second test score based on the second response input (in a manner similar to block 1120), as indicated in block 1126.

The process 1100 can optionally proceed to block 1128 to calculate a visual acuity measurement of the test subject based on the second test score. In some embodiments, the visual acuity measurement can be based on the second test score. In some embodiments, the visual acuity measurement can alternatively or additionally be based on a dimension of the first image and/or a dimension of the second image. In some embodiments, the visual acuity measurement can be a visual acuity score such as a ratio comparing the subject's performance to “normal” vision (e.g., 20/20, 20/40, 20/15, etc.) or other conventional measurements for visual acuity known in the art.

In some embodiments, after calculating a second test score in block 1126, the process 1100 can include displaying a third image based on the second test score (with the third image generally having smaller optotypes), recording a third input, and calculating a third test score before calculating a visual acuity measurement (as in block 1128). Any number of additional iterations of displaying, recording, and scoring can be performed as needed to obtain sufficient testing data to calculate the visual acuity measurement.

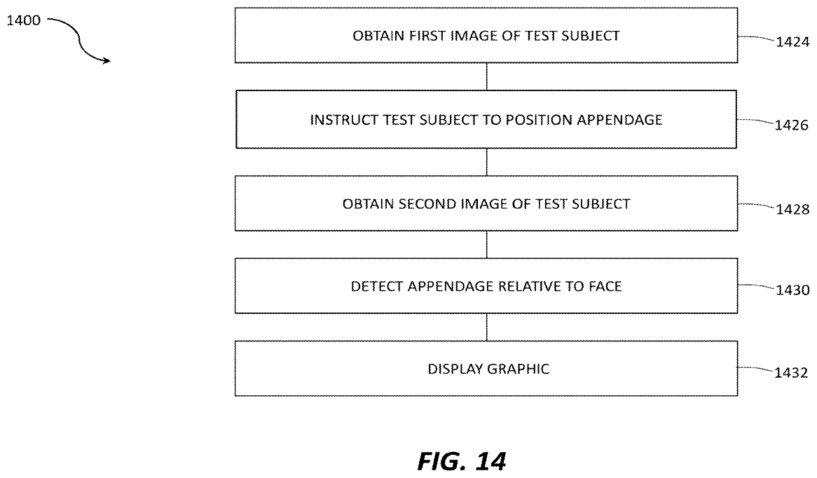

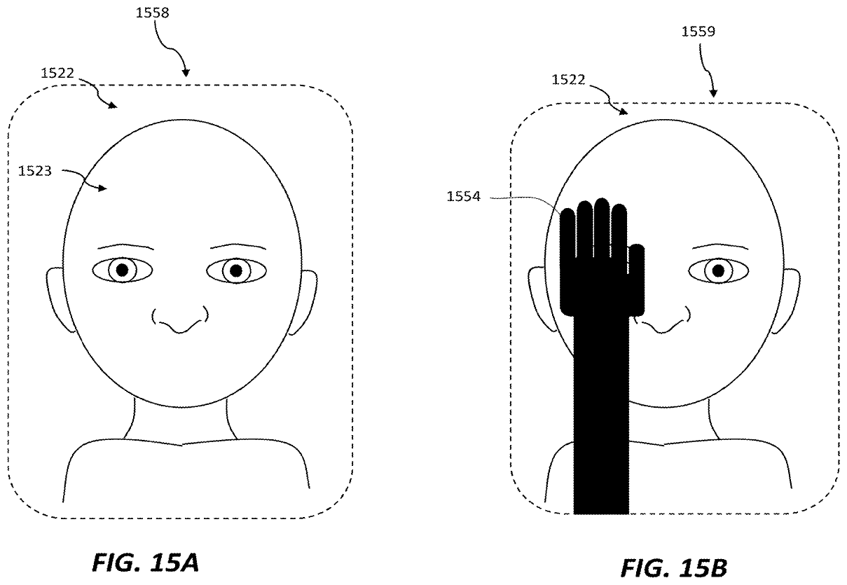

FIGS. 14-15B show aspects of a process 1400 that can be used to administer a visual acuity test using a computing device 202. The process 1400 can include block 1424, wherein the controller obtains an image of a test subject 1522, block 1426, wherein the controller instructs the test subject 1522 to position an appendage 1554, block 1428, wherein the controller obtains a second image of the test subject 1522, and block 1432, wherein the controller displays a graphic 214 to the test subject on a display screen 216 of the computing device 202. In block 1424, the controller can obtain an image 1558 of the test subject 1522 via an image capturing device 218 of a computing device 202. The image can include a face 1523 of the test subject 1522.

In block 1426, the controller can instruct the test subject 1554, by the computing device 202, to position an appendage 1555 of the test subject relative to the face 1523. The appendage 1555 can be part of an arm of the test subject 1522, such as the subject's hand, fingers, or forearm.

In block 1428, the controller can obtain a second image 1559 of the test subject via an image capturing device 218 of a computing device 202.

In block 1430, the controller can detect the appendage 1554 of the test subject 1522 relative to the test subject's face 1523. For example, the controller can execute an object or shape detection algorithm or logic to identify the appendage and face and to verify their locations relative to each other, such as by determining that the appendage is blocking or covering one of the eyes on the face.

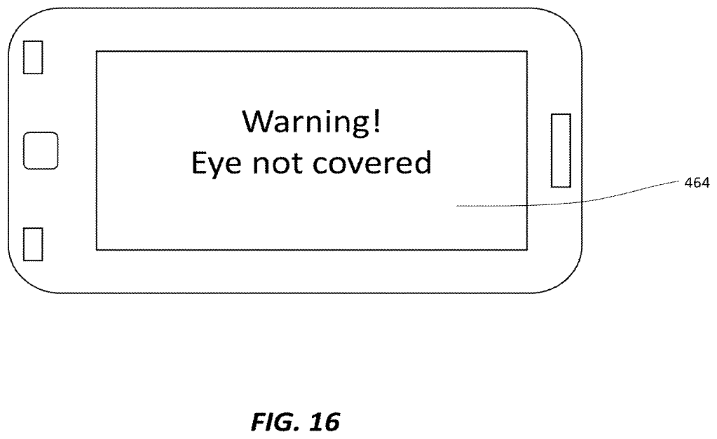

In block 1432, the controller can display to the test subject 1522, by a display screen 216 of the computing device 202, a graphical image 464 (see FIG. 16) in response to detecting the appendage 1555 of the test subject 1522 relative to the face 1523. The graphic can include optotypes for the subject to read while their appendage blocks the vision of one eye. In some embodiments, the process 1400 can further include providing instructions for the subject to move an appendage to block a different eye, verifying that the different eye is blocked, and displaying one or more graphics for the non-blocked eye to read.

Various inventions have been described herein with reference to certain specific embodiments and examples. However, they will be recognized by those skilled in the art that many variations are possible without departing from the scope and spirit of the inventions disclosed herein, in that those inventions set forth in the claims below are intended to cover all variations and modifications of the inventions disclosed without departing from the spirit of the inventions. The terms “including: ” and “having” come as used in the specification and claims shall have the same meaning as the term “comprising.”

1. A method of testing visual acuity of a test subject using a computing device, the method comprising:
obtaining an image of a test subject via an image capturing device of a computing device, the image including a first physical feature of the test subject and a second physical feature of the test subject;
calculating an estimated first size dimension of the first physical feature based on a property of the image capturing device and a first expected size dimension of the first physical feature;
calculating an estimated second size dimension of the second physical feature based on the property of the image capturing device and a second expected size dimension of the second physical feature;
determining a separation distance between the test subject and the image capturing device based on the estimated first size dimension and the estimated second size dimension.

2. The method of claim 1, further comprising displaying a graphic to the test subject using a display screen of the computing device.

3. (canceled)

4. The method of claim 2, wherein a dimension of the graphic on the display screen is based on the separation distance.

5. The method of claim 4, wherein the dimension of the graphic is static while displayed on the display screen.

6. The method of claim 4, wherein the dimension of the graphic is dynamically adjusted while displayed on the display screen based on a second determination of the separation distance between the test subject and the image capturing device.

7. The method of claim 1, wherein the first physical feature includes a portion of an eye of the test subject.

8. (canceled)

9. The method of claim 1, wherein the first expected size dimension is based on:
a mean size dimension of the first physical feature among test subjects having a characteristic in common with the test subject, or
a median size dimension of the first physical feature among test subjects having a characteristic in common with the test subject.

10. (canceled)

11. (canceled)

12. The method of claim 1, further comprising detecting the property of the image capturing device by receiving device reference information from the computing device.

13. (canceled)

14. A method of testing visual acuity of a test subject using a computing device, the method comprising:
determining a separation distance between a test subject and an image capturing device of a computing device;
determining at least one property of a display screen of the computing device;
displaying a graphic to the test subject via the display screen, the graphic having at least one size dimension based on the separation distance and the at least one property of the display screen.

15. (canceled)

16. (canceled)

17. The method of claim 14, wherein the at least one property of the display screen includes an outer dimension or diagonal dimension of a viewable area of the display screen.

18. The method of claim 14, wherein determining the separation distance includes:
obtaining an image of a test subject via the image capturing device of the computing device, the image including a physical feature of the test subject;
calculating an estimated size dimension of the physical feature based on a property of the image capturing device and an expected size dimension of the physical feature;
determining the separation distance based on the estimated size dimension.

19. The method of claim 14, wherein determining the at least one property of the display screen of the computing device includes receiving the at least one property via a signal transmitted from the computing device.

20. The method of claim 14, wherein the at least one size dimension of the graphic is inversely related to the separation distance.

21. The method of claim 14, wherein the at least one size dimension of the graphic is directly related to the at least one property of the display screen.

22. The method of claim 14, wherein the at least one size dimension of the graphic is inversely related to the at least one property of the display screen.

23. The method of claim 14, wherein the image capturing device and the display screen are positioned in a single housing of the computing device.

24. (canceled)

25. A method of testing visual acuity of a test subject using a computing device, the method comprising:
displaying, via a display screen of a computing device, a first graphical image for a test subject, the first graphical image having a first size dimension;
recording, via an input device of the computing device, a response input from the test subject;
calculating a test score based on the response input;
displaying, via the display screen, a second graphical image for the test subject, the second graphical image having a second size dimension, the second size dimension being based on the test score.

26. (canceled)

27. (canceled)

28. The method of claim 25, wherein calculating the test score comprises:
detecting a set of test responses within the response input from the test subject;
comparing each test response of the set of test responses to a set of expected test responses to obtain a set of correct test responses;
comparing a number of correct test responses in the set of correct test responses to a total number of test responses.

29. The method of claim 25, wherein the second size dimension is decreased relative to the first size dimension based on the test score exceeding a threshold score value.

30. The method of claim 25, further comprising:
recording, via the input device, a second response input from the test subject;
calculating a second test score based on the second response input;
calculating a visual acuity measurement of the test subject based on the second test score.

31-32. (canceled)
